(19) United States
(12) Patent Application Publication
(10) Pub. No.: US 2024/0019415 A1
(43) Pub. Date: Jan. 18, 2024

(54) MULTIFUNCTIONAL DETECTOR

(71) Applicant: Xinhua TAN, Yueyang (CN)

(72) Inventor: Xinhua TAN, Yueyang (CN)

(21) Appl. No.: 17/863,944

(22) Filed: Jul. 13, 2022

(51) Int. Cl. G01N 33/24; G01D 21/02

(52) U.S. Cl. CPC G01N 33/246; G01D 21/02

(57) ABSTRACT

The present disclosure discloses a multifunctional detector including a soil tester and a microcontroller disposed on the soil tester, wherein the microcontroller is connected with a PH acquisition unit, the PH acquisition unit is connected with a humidity sensor, the microcontroller is connected with an acquired data processing system, and the acquired data processing system includes a temperature sensor, a button module, a display module, a clock module, a storage module, a communication module, a calibration module, an environmental data acquisition module, an environmental light intensity acquisition module, a probe data acquisition module and a probe data receiving module. The multifunctional detector is accurate in measurement, high in stability, good in reliability, rapid and convenient in use, more in measurement type, more convenient, more stable and capable of separately storing data, the power consumption of a hardware circuit of a control system is lower, and a probe is detachable.

### TECHNICAL FIELD

The present disclosure relates to the technical field of soil detection, in particular to a multifunctional detector.

### BACKGROUND

The acidity or alkalinity (PH), temperature and humidity of soil are important factors affecting the growth of plants, and particularly, the PH of the soil will directly affect the growth of the plants. The acquisition of the PH of the soil is an important link before planting, and the humidity of the soil is one of important factors indicating the normal growth of crops during growth of the plants.

In the prior art, the temperature and PH value of the soil may not be displayed at the same time, great measurement errors are brought, the test function is relatively single, a probe is undetachable, test data may not be stored, and therefore, we propose a multifunctional detector to solve the above-mentioned problems.

### SUMMARY

A purpose of the present disclosure is to provide a multifunctional detector to solve the problems proposed in the background art.

For achieving the above-mentioned purpose, the present disclosure provides the following technical solutions: a multifunctional detector, comprising a soil tester and a microcontroller disposed on the soil tester, wherein the microcontroller is connected with a PH acquisition unit, the PH acquisition unit is connected with a humidity sensor, the microcontroller is connected with an acquired data processing system, and the acquired data processing system comprises a temperature sensor, a display module, a storage module, an environmental data acquisition module, an environmental light intensity acquisition module, a probe data acquisition module and a probe data receiving module;
wherein the PH acquisition unit comprises a measuring instrument PH for measuring the PH of soil, the measuring instrument PH is connected with a resistor R6 by a line, the resistor R6 is connected with a non-inverting input end of a first operational amplifier, an inverting input end of the first operational amplifier is connected to a connecting line of an output end of the first operational amplifier by a line, the other end of the connecting line of the output end of the operational amplifier is connected with a resistor R11, the resistor R11 is connected with an inverting input end of a second operational amplifier, the inverting input end of the second operational amplifier and an output end of the second operational amplifier are connected in parallel with a resistor R10, a resistor R9 and a VCC are connected to a connecting line of a non-inverting input end of the second operational amplifier, a connecting line, connected with the resistor R9, of the non-inverting input end of the second operational amplifier is connected with a resistor R12 and is grounded, the output end of the second operational amplifier is connected with a resistor R13, the other end of the resistor R13 is connected with the microcontroller, and a connecting line, connected with the microcontroller, of the resistor R13 is connected with a capacitor C2 and is grounded.

Further, the microcontroller is a PIC18F series single chip microcomputer with low power consumption, the microcontroller is used for processing data, is a 12-bit high-precision processor and comprises a chip U1, and the chip U1 is of the type N76E003AQ20.

Further, the probe data acquisition module comprises a USB2, the USB2 is connected in parallel with a NTC and is grounded, the USB2 is grounded, and the USB2 is of the type TYPE-C-31-G-06.

Further, the probe data receiving module comprises a USB1 connected with an end TM of the chip U1, the USB1 is connected in parallel with a resistor R9, a resistor R11, a resistor R8, a capacitor C15, a resistor R5, a voltage-stabilizing diode ZD1 and a capacitor C16, a voltage which is +3.3 is connected to a connecting line, connected with the resistor R11, of the resistor R9, the USC1 is grounded, a connecting line, connected with the voltage-stabilizing diode ZD1, of the resistor R5 is connected with an end TM/PH of the chip U1, and the USC1 is of the type TYPE-C 6P LT6.8(073).

Further, the environmental data acquisition module comprises a resistor R25 with one end being connected with an end P01 of the chip U1, the resistor R25 is connected with an environmental temperature SE1, the environmental temperature SE1 is connected in parallel with a resistor R26, a connecting line, connected with the resistor R26, on the environmental temperature SE1 is connected with a resistor R24 and is connected with an end P03 of the chip U1, and a connecting line, connected with the resistor R26, on the environmental temperature SE1 is connected with an end SM of the chip UL.

Further, the display model is of the type LCD-30P.

Further, the temperature sensor is used for acquiring humidity data information.

Compared with the prior art, the present disclosure has the beneficial effects that
1. the multifunctional detector is accurate in measurement, high instability, good in reliability and rapid and convenient to use;
2. the power consumption of a hardware circuit of a control system is lower;
3. the multifunctional detector is more in measurement type, more convenient and more stable;
4. the probe is detachable, data is separately stored, the probe is switchable and capable of wirelessly transmitting data and achieving automatic recognition; and
5. in addition to the PH value, the humidity and temperature of the soil and the AQI may be further measured.

### BRIEF DESCRIPTION OF DRAWINGS

FIG. 1 is a schematic diagram of modules in the present disclosure;

FIG. 2 is a schematic circuit diagram of a PH acquisition unit in the present disclosure;

FIG. 3 is a schematic diagram of a connecting circuit of a microcontroller in the present disclosure;

FIG. 4 is a schematic connection diagram of a probe and a detector in the present disclosure;

FIG. 5 is a schematic diagram of the probe and the detector in the present disclosure;

FIG. 6 is a schematic circuit diagram of the microcontroller in the present disclosure;

FIG. 7 is a schematic circuit diagram of an environmental data acquisition module in the present disclosure;

FIG. 8 is a schematic circuit diagram of a probe data acquisition module in the present disclosure; and

FIG. 9 is a schematic circuit diagram of a probe data receiving module in the present disclosure.

### DETAILED DESCRIPTION OF THE EMBODIMENTS

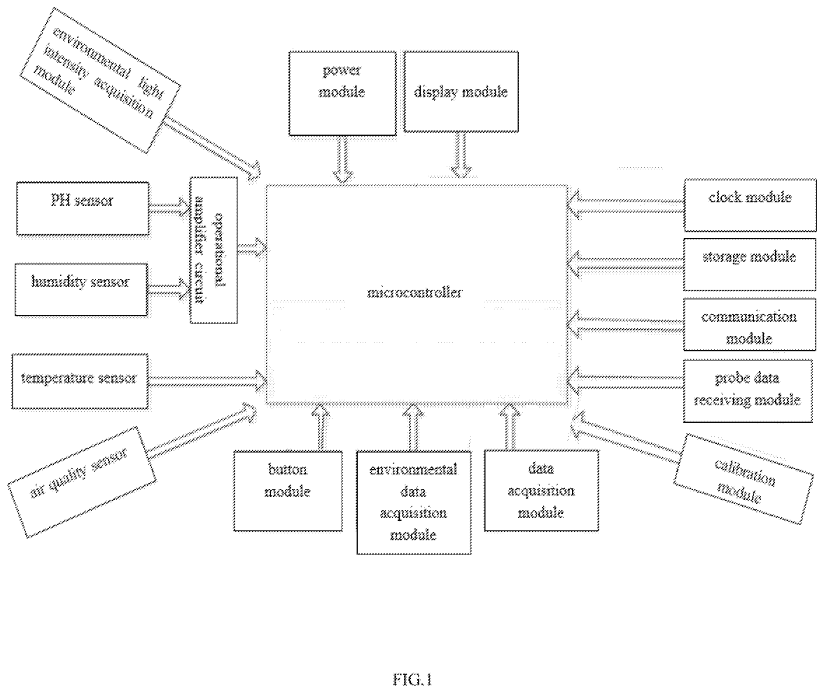

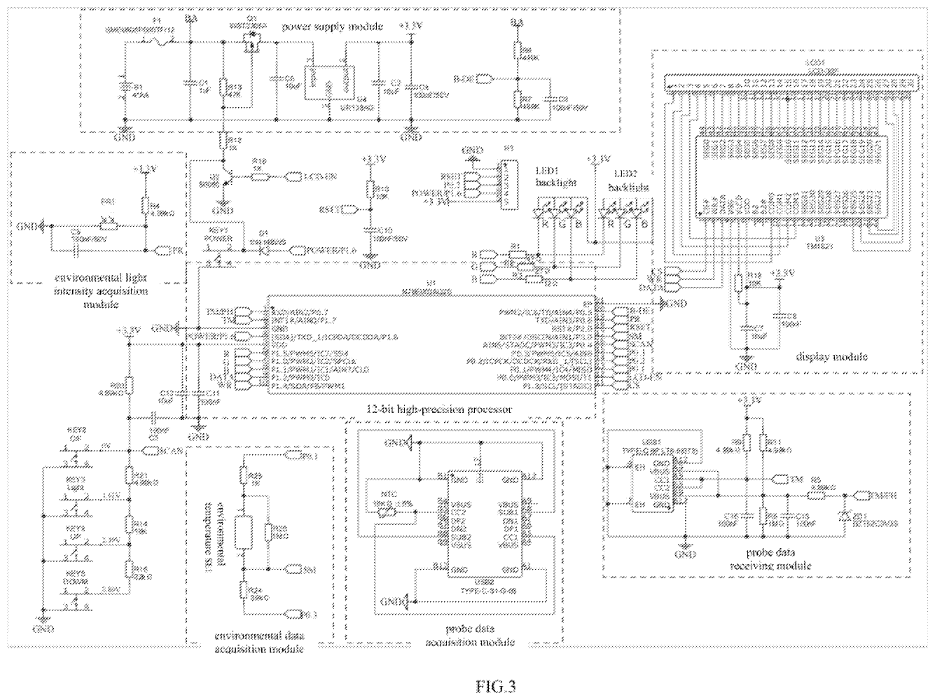

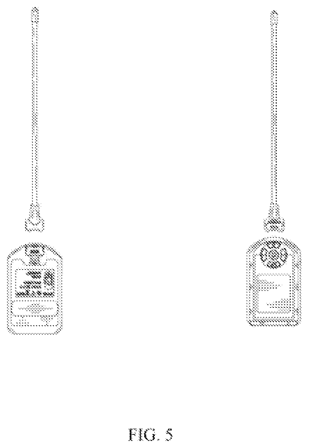

With reference to FIGS. 1 to 9, the present disclosure provides a technical solution in which a multifunctional detector includes a soil tester and a microcontroller disposed on the soil tester;

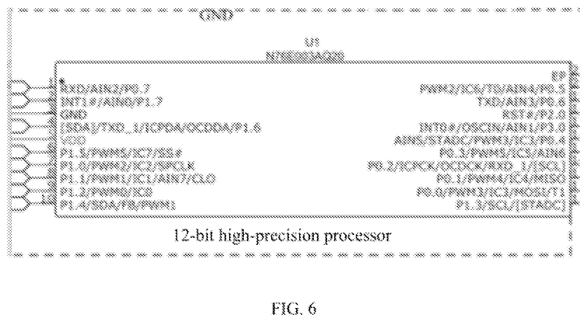

As shown in FIG. 6,
the microcontroller is a PIC18F series single chip microcomputer with low power consumption, the microcontroller is used for processing data, is a 12-bit high-precision processor and includes a chip U1, and the chip U1 is of the type N76E003AQ20;
the microcontroller is connected with a PH acquisition unit, the PH acquisition unit is connected with a humidity sensor, the microcontroller is connected with an acquired data processing system, and the acquired data processing system includes a temperature sensor for acquiring humidity data information, a button module, a display module of the type LCD-30P, a clock module, a storage module, a communication module, a calibration module, an environmental data acquisition module, an environmental light intensity acquisition module, a probe data acquisition module and a probe data receiving module;

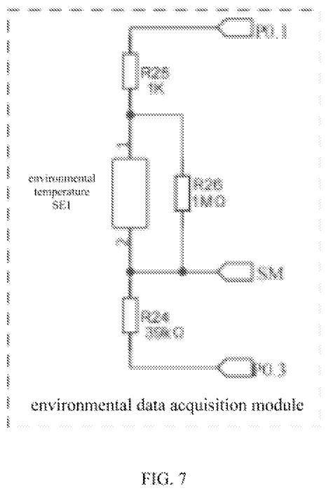

As shown in FIG. 7,
the environmental data acquisition module includes a resistor R25 with one end being connected with an end P01 of the chip U1, the resistor R25 is connected with an environmental temperature SE1, the environmental temperature SE1 is connected in parallel with a resistor R26, a connecting line, connected with the resistor R26, on the environmental temperature SE1 is connected with a resistor R24 and is connected with an end P03 of the chip U1, and a connecting line, connected with the resistor R26, on the environmental temperature SE1 is connected with an end SM of the chip U1;

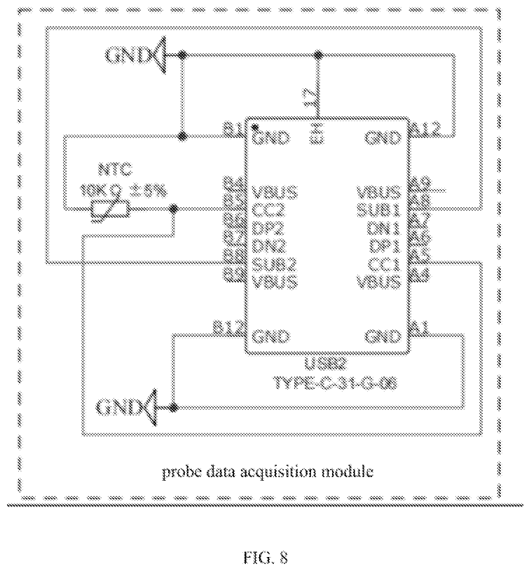

As shown in FIG. 8,
the probe data acquisition module includes a USB2, the USB2 is connected in parallel with a NTC and is grounded, the USB2 is grounded, and the USB2 is of the type TYPE-C-31-G-06;

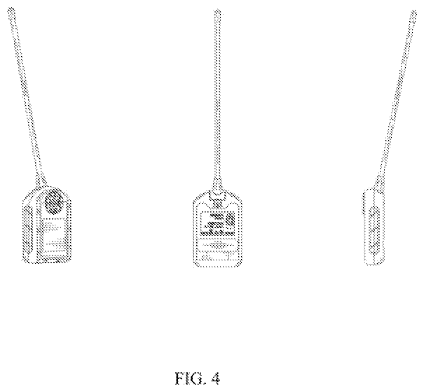

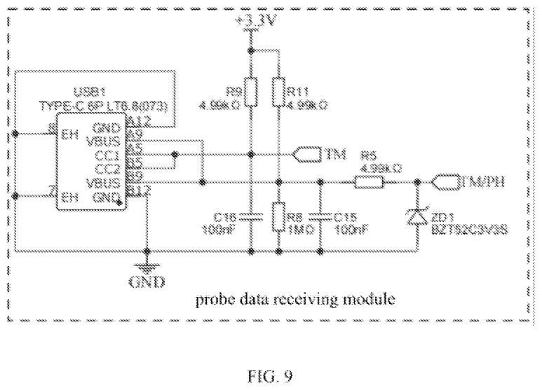

As shown in FIG. 9,
the probe data receiving module includes a USB1 connected with an end TM of the chip U1, the USB1 is connected in parallel with a resistor R9, a resistor R11, a resistor R8, a capacitor C15, a resistor R5, a voltage-stabilizing diode ZD1 and a capacitor C16, a voltage which is +3.3 is connected to a connecting line, connected with the resistor R11, of the resistor R9, the USC1 is grounded, a connecting line, connected with the voltage-stabilizing diode ZD1, of the resistor R5 is connected with an end TM/PH of the chip U1, the USC1 is of the type TYPE-C 6P LT6.8(073), as shown in FIG. 4, the probe data acquisition module is connected with the probe data receiving module in a TYPE-C splicing manner;
wherein the PH acquisition unit includes a measuring instrument PH for measuring the PH of soil, the measuring instrument PH is connected with a resistor R6 by a line, the resistor R6 is connected with a non-inverting input end of a first operational amplifier, an inverting input end of the first operational amplifier is connected to a connecting line of an output end of the first operational amplifier by a line, the other end of the connecting line of the output end of the operational amplifier is connected with a resistor R11, the resistor R11 is connected with an inverting input end of a second operational amplifier, the inverting input end of the second operational amplifier and an output end of the second operational amplifier are connected in parallel with a resistor R10, a resistor R9 and a VCC are connected to a connecting line of a non-inverting input end of the second operational amplifier, a connecting line, connected with the resistor R9, of the non-inverting input end of the second operational amplifier is connected with a resistor R12 and is grounded the output end of the second operational amplifier is connected with a resistor R13, the other end of the resistor R13 is connected with the microcontroller, and a connecting line, connected with the microcontroller, of the resistor R13 is connected with a capacitor C2 and is grounded.

PH Signal Acquisition Circuit

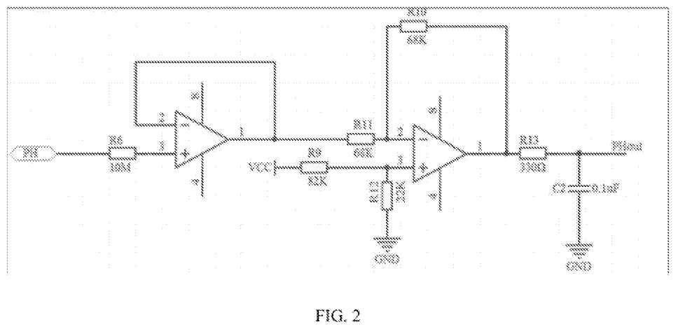

With reference to FIG. 2, a circuit diagram of the PH acquisition unit is shown as FIG. 2 in which the letter PH represents a voltage signal returned by a PH sensor, the voltage signal firstly passes by a voltage follower, the second stage of the circuit is a subtraction operation circuit, a signal of which the voltage is about 1V is subtracted by the voltage signal of the PH sensor, it may be known from table I that the voltage change of the PH sensor may be positive or negative, a voltage signal which is greater than zero may only be acquired during AD sampling of the PIC18F single chip microcomputer. When the voltage signal passes by a second-stage circuit, and if the PH is equal to 0. PHout is about 0.6 V, if the PH is equal to 14. PHout is about 1.4 V; after the signal of the sensor passes by the circuit as shown in the figure, the signal is wholly increased to be greater than zero, and the PH and the voltage are in a positive relationship, and an output range of PHout is that the signal may be normally acquired by AD. The capacitor C2 is used for eliminating interference to the input end.

Humidity Signal Acquisition Circuit

The humidity signal acquisition circuit adopts a voltage division manner, and the voltage change is directly acquired by AD of the single chip microcomputer. The resistors R1 and R3 play a voltage division role, the resistors R4 and R5 play a voltage division role, and C4 plays a filtration role to avoid influences from interference signals.

10 groups of soil samples which are different in PH and humidity are prepared in a 25° C. laboratory, an exact value of PH is a numerical value measured by a PHS-3C acidity meter, the PH value measured by using a soil PH and humidity measuring instrument is a measured value, an exact value of the percentage of humidity is a calculated value obtained before and after the 10 groups of soil samples are dried, a humidity value measured by the soil PH and humidity measuring instrument is a measured value, the PHS-3C acidity meter and the soil PH and humidity measuring instrument are respectively inserted to the 10 groups of samples to perform measuring and recording, then, the 10 groups of soil samples are dried and weighed by a balance, and the humidity is calculated and recorded.

The soil measured in this experiment is has the PH mainly centralized at 4 to 10 and the humidity centralized at 3% to 18%. In an actual farmland, the PH and humidity of the soil have practical significance in the above-mentioned range. It may be known by an experimental measurement result that the PH error of the soil is less than 0.1 PH, the humidity error is less than 0.5%, and thus, the production requirement of a product is met.

Measurement for PH Value of Soil by Using Combined Bipolar Potential Difference Method

ΔE=ΔPH*59.16*(273.15+T)/298.15 Expression:

In the expression, ΔE: bipolar potential difference of combined electrode
ΔPH: difference of PH change of solution
T: current temperature

At the room temperature which is 25° C., a relationship between a voltage output by a standard PH sensor and the PH value is shown as follows: when the PH is changed to 1, the voltage change is 59.16 mV, and when PH>7, the bipolar potential difference of the PH sensor is less than zero: when PH=7, the bipolar potential difference of the PH sensor is equal to 0; and when PH<7, the bipolar potential difference of the PH sensor is greater than 0, the PH value and the voltage are in a relationship of inverse proportion, however, if an electrode sensor is used for a long term or the temperature is changed, such a corresponding relationship is not met, and therefore, certain compensation and regular calibration are needed. The single chip microcomputer AD used by us may only acquire a voltage value ranging from 0 V to 5 V, and therefore, a voltage returned by the PH sensor and a positive voltage are subtracted by a subtracter to ensure that normal acquisition may be performed by AD.

Measurement for Humidity of Soil by Using Resistive Voltage Division Method

Within a certain range (the soil contains 0% to 18% of water), the greater the percentage of the water in the soil is, the stronger the conductivity of the soil is. At the moment, the soil is equivalent to high resistance, the change of the percentage of the water in the soil causes the change of the conductivity of the soil, and therefore, the content of the water in the soil may be roughly calculated by using a resistive voltage division method.

U=U0*Rx/(Rx+RO) Expression:

In the expression,
U: voltage division output voltage
U0: voltage division output voltage
Rx: soil resistance
R0: voltage division resistance.

The multifunctional detector is accurate in measurement, high in stability, good in reliability and rapid and convenient to use:
the power consumption of a hardware circuit of a control system is lower;
the multifunctional detector is accurate in measurement, high in stability, good in reliability and rapid and convenient to use;
the power consumption of a hardware circuit of a control system is lower;
the multifunctional detector is more in measurement type, capable of more accurately storing data, more convenient and more stable; and
the probe may be separated and detached from the detector, and the detached probe may separately test the quality of water and the quality of soil; and when the probe is combined with the detector, in addition to the basic function of measuring the PH values of the soil and the water, the humidity and temperature of the soil and the AQI may be further measured, and the detected data may be stored separately.

What is claimed is:

1. A multifunctional detector, comprising a soil tester and a microcontroller disposed on the soil tester, wherein the microcontroller is connected with a PH acquisition unit, the PH acquisition unit is connected with a humidity sensor, the microcontroller is connected with an acquired data processing system, and the acquired data processing system comprises a temperature sensor, a display module, a storage module, an environmental data acquisition module, an environmental light intensity acquisition module, a probe data acquisition module and a probe data receiving module;
wherein the PH acquisition unit comprises a measuring instrument PH for measuring the PH of soil, the measuring instrument PH is connected with a resistor R6 by a line, the resistor R6 is connected with a non-inverting input end of a first operational amplifier, an inverting input end of the first operational amplifier is connected to a connecting line of an output end of the first operational amplifier by a line, the other end of the connecting line of the output end of the operational amplifier is connected with a resistor R11, the resistor R11 is connected with an inverting input end of a second operational amplifier, the inverting input end of the second operational amplifier and an output end of the second operational amplifier are connected in parallel with a resistor R10, a resistor R9 and a VCC are connected to a connecting line of a non-inverting input end of the second operational amplifier, a connecting line, connected with the resistor R9, of the non-inverting input end of the second operational amplifier is connected with a resistor R12 and is grounded, the output end of the second operational amplifier is connected with a resistor R13, the other end of the resistor R13 is connected with the microcontroller, and a connecting line, connected with the microcontroller, of the resistor R13 is connected with a capacitor C2 and is grounded.

2. The multifunctional detector of claim 1, wherein the microcontroller is a PIC18F series single chip microcomputer with low power consumption, the microcontroller is used for processing data, is a 12-bit high-precision processor and comprises a chip U1, and the chip U1 is of the type N76E003AQ20.

3. The multifunctional detector of claim 1, wherein the probe data acquisition module comprises a USB2, the USB2 is connected in parallel with a NTC and is grounded, the USB2 is grounded, and the USB2 is of the type TYPE-C-31-G-06.

4. The multifunctional detector of claim 1, wherein the probe data receiving module comprises a USB1 connected with an end TM of the chip U1, the USB1 is connected in parallel with a resistor R9, a resistor R11, a resistor R8, a capacitor C15, a resistor R5, a voltage-stabilizing diode ZD1 and a capacitor C16, a voltage which is +3.3 is connected to a connecting line, connected with the resistor R11, of the resistor R9, the USC1 is grounded, a connecting line, connected with the voltage-stabilizing diode ZD1, of the resistor R5 is connected with an end TM/PH of the chip U1, and the USC1 is of the type TYPE-C 6P LT6.8(073).

5. The multifunctional detector of claim 4, wherein the environmental data acquisition module comprises a resistor R25 with one end being connected with an end P01 of the chip U1, the resistor R25 is connected with an environmental temperature SE1, the environmental temperature SE1 is connected in parallel with a resistor R26, a connecting line, connected with the resistor R26, on the environmental temperature SE1 is connected with a resistor R24 and is connected with an end P03 of the chip U1, and a connecting line, connected with the resistor R26, on the environmental temperature SE1 is connected with an end SM of the chip U1.

6. The multifunctional detector of claim 1, wherein the display model is of the type LCD-30P.

7. The multifunctional detector of claim 1, wherein the temperature sensor is used for acquiring humidity data information.
